(19) United States
(12) Patent Application Publication
(10) Pub. No.: US 2023/0157611 A1
(43) Pub. Date: May 25, 2023

(54) WEAK CURRENT SIGNAL ACQUISITION CIRCUIT, AND SEAT AND SEAT COVER HAVING SAME

(71) Applicants: Guangzhou Eaphonetech Co., Ltd., Guangdong (CN); Guangzhou Xinxiangsui Technology Co., Ltd., Guangdong (CN)

(72) Inventors: Pei Wang, Guangdong (CN); Jinhe Luo, Guangdong (CN)

(21) Appl. No.: 17/920,380

(22) PCT Filed: Mar. 30, 2021
(86) PCT No.: PCT/CN2021/083998

§ 371 Date: Oct. 20, 2022

(30) Foreign Application Priority Data
Apr. 20, 2020 (CN) 202010315692.4

(51) Int. Cl. A61B 5/305; A47K 13/24; A61B 5/282; A61B 5/308; A61B 5/00

(52) U.S. Cl. CPC A61B 5/305; A47K 13/24; A61B 5/282; A61B 5/308; A61B 5/6887

(57) ABSTRACT

A weak current signal acquisition circuit, and a seat (10) and a seat cover (100) having the circuit. The weak current signal acquisition circuit uses two signal acquisition units as front input electrodes of an amplification circuit, and uses an in-phase differential input mode, so that the ratio of amplitudes of a differential mode signal sent to a subsequent-stage operational amplifier to a common mode signal is improved, thereby achieving the purpose of improving the signal-to-noise ratio of a weak current signal extracted by a signal acquisition unit. When people use a seat cover (100) in which a weak current signal acquisition circuit is mounted, thighs are in contact with a signal acquisition unit provided on the seat (10), such that the weak current signal acquisition circuit can acquire a human body's electrocardiosignal more effectively.

### TECHNICAL FIELD

The present disclosure relates to the field of toilet seat technologies, and specifically to a weak electrical signal acquisition circuit, and a seat ring and a toilet seat each having the weak electrical signal acquisition circuit.

### BACKGROUND

A closestool, also known as a toilet, is a necessary fixture in people's daily life, and toilet seats with medical and health care functions are rather popular. At present, smart toilet seats are able to collect biometric signals from human bodies, such as electrocardiographic (ECG) signals and electrical impedances of human bodies, to monitor the health conditions of human bodies. However, in a dry winter, the skin surface of the thigh has a rather high resistance value, which makes it extremely difficult to acquire ECG signals via the thigh. In addition, the acquired ECG signals are rather weak, which may affect the subsequent processing of the ECG signals.

### SUMMARY

In view of this, it is desired to provide a weak electrical signal acquisition circuit with improved common mode rejection ratio and signal-to-noise ratio (in acquired signals), and also a toilet seat having the weak electrical signal acquisition circuit.

Provided is a weak electrical signal acquisition circuit, which includes two signal acquisition units that include two signal receiving electrode sheets and two operational amplifiers, where the two operational amplifiers form a differential circuit, the differential circuit further including a first feedback resistor R₃, a second feedback resistor R₃′, and a negative feedback resistor R₄ provided between the first feedback resistor R₃ and the second feedback resistor R₃′, where the negative feedback resistor R₄ serves as a negative feedback circuit for filtering differential mode signals received by the signal receiving electrode sheets.

Furthermore, the two signal receiving electrode sheets include a first signal receiving electrode sheet and a second signal receiving electrode sheet, the two operational amplifiers include a first operational amplifier A and a second operational amplifier A′, and the two signal acquisition units include a first signal acquisition unit and a second signal acquisition unit, wherein

the first signal receiving electrode sheet and the first operational amplifier A form the first signal acquisition unit, the first signal receiving electrode sheet being connected to a non-inverting input terminal of the first operational amplifier A, and the first feedback resistor R₃ being provided between an inverting input terminal and an output terminal of the first operational amplifier A; and

the second signal receiving electrode sheet and the second operational amplifier A′ form the second signal acquisition unit, the second signal receiving electrode sheet being connected to a non-inverting input terminal of the second operational amplifier A′, and the second feedback resistor R₃ ′ being provided between an inverting input terminal and an output terminal of the second operational amplifier A′.

Furthermore, the two signal receiving electrode sheets are active electrode sheets, and are configured to receive common mode signals and differential mode signals.

Furthermore, the negative feedback circuit is formed by a single resistor or a network assembly consisting of a plurality of electronic components and having a frequency characteristic; or the negative feedback circuit includes a network assembly having a frequency characteristic of full pass, low pass, high pass, band pass, or band resistance.

In addition, provided is a seat ring having a weak electrical signal acquisition circuit, where the seat ring is fitted with the weak electrical signal acquisition circuit as described above.

Furthermore, the seat ring has two mounting holes respectively provided near tops of upper surfaces of left and right portions thereof, with the two signal acquisition units respectively provided in the two mounting holes, wherein the two mounting holes have openings respectively covered by the two signal receiving electrode sheets, with the first operational amplifier A and the second operational amplifier A′ respectively mounted in the two mounting holes.

Furthermore, the seat ring has two conductor through-holes respectively provided in the left and right portions, the two conductor through-holes having one ends provided respectively at bottoms of the two mounting holes and the other ends each provided at a connection portion between the seat ring and a seat lid, wherein the two conductor through-holes have two signal transmission lines respectively provided therein, the two signal transmission lines having one ends respectively connected to output terminals of the first operational amplifier A and the second operational amplifier A′ and the other ends each connected to a secondary differential amplifier circuit.

Furthermore, each of the two signal transmission lines is in a form of a shielded cable.

In addition, a toilet seat having a weak electrical signal acquisition circuit is provided, which includes a seat ring having the weak electrical signal acquisition circuit as described above, where the seat ring further includes a seat ring adjustment mechanism that is provided under the seat ring and configured to lift a front end of the seat ring by a certain angle.

The present disclosure has following advantageous technical effects.

1. According to the present disclosure, the signal acquisition unit has two active electrode sheets connected to inverting input terminals of the differential amplifiers interconnected by the negative feedback resistor R₄, such that the differential gain of common mode signals will not be amplified. As a result, the common mode saturation can be prevented from occurring in the circuit, and the common mode rejection ratio of the whole circuit can also be improved, thereby improving the signal-to-noise ratio of the system.

2. According to the present disclosure, the common mode current flowing through the negative feedback resistor R₄ is zero. That is, for the common mode signal, the negative feedback resistor R₄ is equivalent to an open circuit. Thus, the characteristics of the negative feedback resistor R₄ are independent of the common mode signal, which means that the negative feedback resistor R₄ may be replaced with a network assembly having a frequency characteristic such as full pass, low pass, high pass, band pass, or band resistance.

3. According to the present disclosure, the seat ring adjustment mechanism added to the seat ring enables a tilt angle of the seat ring to be adjustable, such that the human body can be in more close contact with the signal acquisition unit, and thus accuracy of ECG signals collected from the human body can be further improved.

### BRIEF DESCRIPTION OF THE DRAWINGS

FIG. 1 is a first schematic structural diagram of a weak electrical signal acquisition circuit according to an embodiment of the present disclosure;

FIG. 2 is a second schematic structural diagram of a weak electrical signal acquisition circuit according to an embodiment of the present disclosure;

FIG. 3 is a third schematic structural diagram of a weak electrical signal acquisition circuit according to an embodiment of the present disclosure;

FIG. 4 is a schematic structural diagram of a seat ring of a toilet seat having a weak electrical signal acquisition circuit according to an embodiment of the present disclosure;

FIG. 5 is a schematic structural diagram of a first way of connection of signal transmission lines in the weak electrical signal acquisition circuit of the toilet seat having the weak electrical signal acquisition circuit according to an embodiment of the present disclosure;

FIG. 6 is a schematic structural diagram of a second way of connection of signal transmission lines in the weak electrical signal acquisition circuit of the toilet seat having the weak electrical signal acquisition circuit according to an embodiment of the present disclosure; and

FIG. 7 is a schematic structural diagram of a toilet seat having the weak signal acquisition circuit and a foot rest according to an embodiment of the present disclosure.

### DETAILED DESCRIPTION

The weak electrical signal acquisition circuit, and the seat ring and toilet seat having the weak electrical signal acquisition circuit are taken as an example of this embodiment, and the present disclosure will be further illustrated in details with reference to the specific embodiments and accompanying drawings.

### EXAMPLE 1

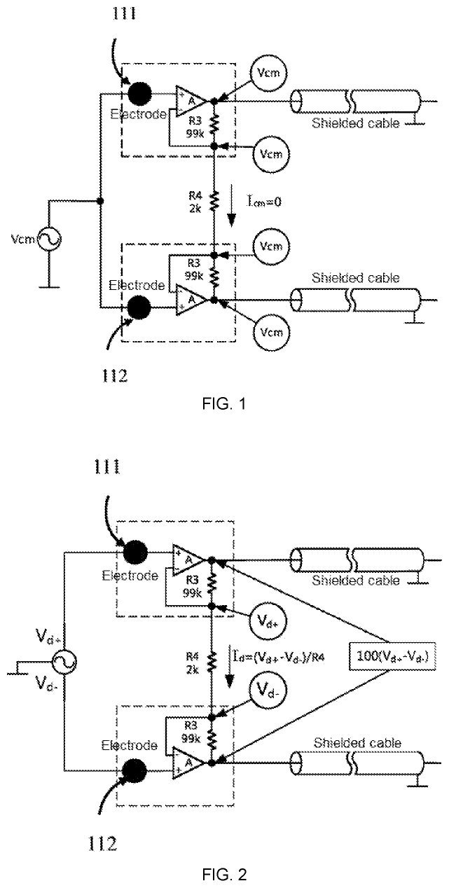

As shown in FIGS. 1, 2 and 3, a weak electrical signal acquisition circuit according to embodiments of the present disclosure includes two signal acquisition units that include two signal receiving electrode sheets and two operational amplifiers. The two operational amplifiers form a differential circuit. The differential circuit further includes a first feedback resistor R₃, a second feedback resistor R₃′, and a negative feedback resistor R₄ provided between the first feedback resistor R₃ and the second feedback resistor R₃′.

Furthermore, the two signal receiving electrode sheets include a first signal receiving electrode sheet 111 and a second signal receiving electrode sheet 112, the two operational amplifiers include a first operational amplifier A and a second operational amplifier A′, and the two signal acquisition units include a first signal acquisition unit and a second signal acquisition unit.

The first signal receiving electrode sheet 111 and the first operational amplifier A form the first signal acquisition unit, where the first signal receiving electrode sheet 111 is connected to a non-inverting input terminal of the first operational amplifier A and the first feedback resistor R₃ is provided between an inverting input terminal and an output terminal of the first operational amplifierA.

The second signal receiving electrode sheet 112 and the second operational amplifier A′ form the second signal acquisition unit, where the second signal receiving electrode sheet 112 is connected to a non-inverting input terminal of the second operational amplifier A′, and the second feedback resistor R₃ ′ is provided between an inverting input terminal and an output terminal of the second operational amplifierA'.

Furthermore, the two signal receiving electrode sheets are active electrode sheets, and are configured to receive common mode signals and differential mode signals.

Specifically, referring to FIGS. 1 and 2, the negative feedback resistor R₄ is common to inverting input terminals of the first operational amplifier A and the second operational amplifier A′.

If a common mode signal Vcm is input to the two active electrodes simultaneously, a potential difference generated across the negative feedback resistor R₄ is 0 and thus a common mode current lcm generated at the negative feedback resistor R₄ is 0. Thus, the differential circuit formed by the first operational amplifier A and the second operational amplifier A′ has no amplifying effect on the common mode signal Vcm. That is, the common mode gain satisfies:

G_{c}=1

If a differential mode signal V_{d} is input to the two active electrodes, the differential mode current flowing through the negative feedback resistor R₄ satisfies:

I_{d}=(V_{d+}−V_{d-})/R₄

The differential gain satisfies:

G_{d}=(2R₃+R₄)/R₄

Based on the aforesaid analysis, it can be seen that the negative feedback resistor R₄ is common to the inverting input terminals of the first operational amplifier A and the second operational amplifier A′, which can improve the differential gain without amplifying the common mode signal, and thereby prevent the common mode saturation from occurring in the circuit and improve the signal-to-noise ratio of the circuit. In addition, it can be seen that the common mode rejection ratio of the circuit is CMRR=G_{d}/G_{c}=G_{d}.

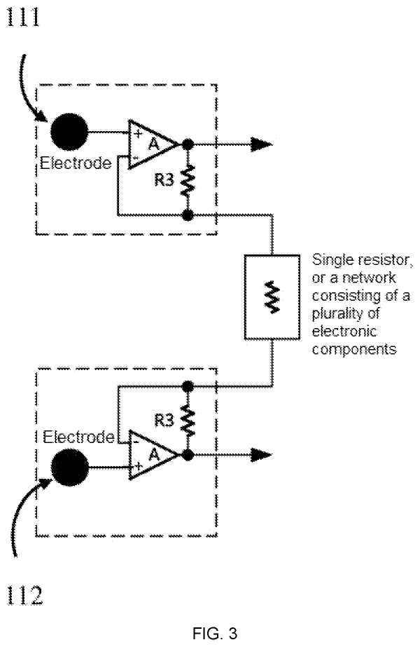

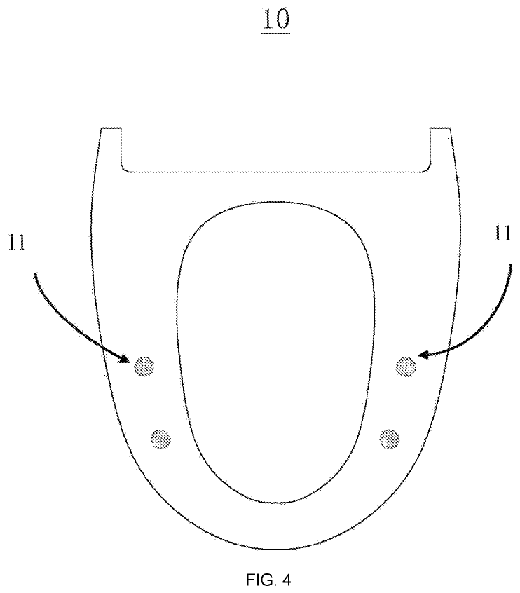

Specifically, referring to FIG. 3, the common mode current l_{cm} flowing through the negative feedback resistor R₄ is 0, which is equivalent to an open circuit for the common mode signal, thus the frequency characteristic of the negative feedback resistor

R₄ is independent of the common mode signal. Therefore, the negative feedback resistor R₄ may be replaced by a network assembly that has a frequency characteristic, where the network assembly is purely resistive to the common mode signal while has a filtering function for the differential mode signal.

Furthermore, the negative feedback resistor R₄ serves as a negative feedback circuit for filtering the differential mode signals received by the signal receiving electrode sheets.

Furthermore, the negative feedback circuit is formed by a single resistor, or a network assembly consisting of a plurality of electronic components and having a frequency characteristic; or the negative feedback circuit includes a network assembly having a frequency characteristic of full pass, low pass, high pass, band pass, or band resistance.

In this embodiment, the two signal acquisition units serve as pre-input electrodes of an amplification circuit and adopt the in-phase differential input mode, such that the amplitude ratio of the differential mode signals to the common mode signals as sent to the post-operational amplifier is increased, and the purpose of improving the signal-to-noise ratio in the weak electrical signal acquired by the signal acquisition unit can thereby be achieved.

### EXAMPLE 2

As shown in FIG. 4, a seat ring 10 having a weak electrical signal acquisition circuit according to embodiments of the present disclosure is fitted with the weak electrical signal acquisition circuit as described above.

Furthermore, the seat ring 10 has two mounting holes 11 respectively provided near tops of upper surfaces of left and right portions thereof, with the two signal acquisition units respectively provided in the two mounting holes 11, wherein the two mounting holes 11 have openings respectively covered by the two signal receiving electrode sheets, with the first operational amplifier A and the second operational amplifier A′ respectively mounted in the two mounting holes 11.

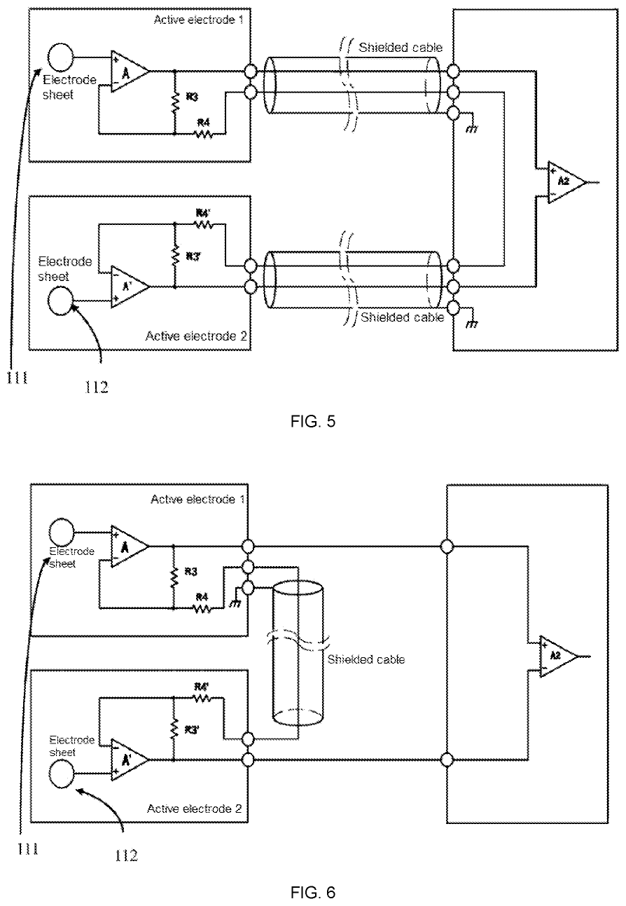

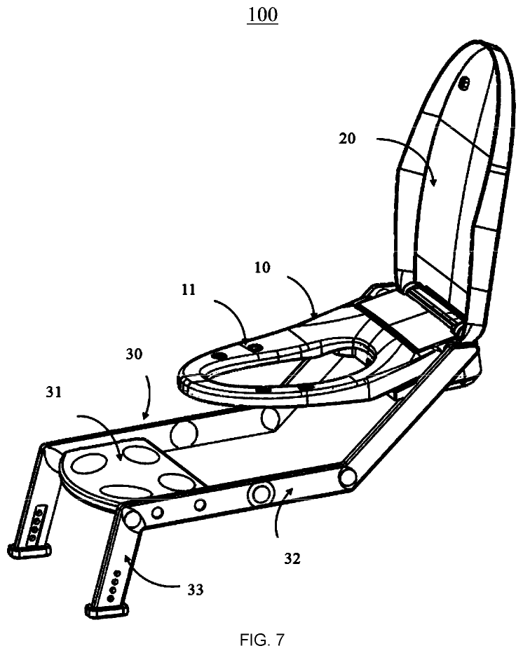

Furthermore, referring to FIGS. 5 and 6, the seat ring 10 has two conductor through-holes respectively provided in the left and right portions. The two conductor through-holes having one ends provided respectively at bottoms of the two mounting holes 11 and the other ends each provided respectively at the connection portion between the seat ring 10 and a seat lid 20. In addition, the two conductor through-holes have two signal transmission lines respectively provided therein, the two signal transmission lines having one ends respectively connected to output terminals of the first operational amplifier A and the second operational amplifier A′ and the other ends each connected to a secondary differential amplifier circuit. Furthermore, each of the two signal transmission lines is in a form of a shielded cable.

Specifically, the seat ring 10 fitted with the weak electrical signal acquisition circuit according to this embodiment may be mounted either on a normal toilet seat without heating and flushing functions or on a smart toilet seat with heating and flushing functions and functions of detecting human physiological parameters (e.g. body fat parameters, weight parameters, ECG signals, etc.).

Specifically, the first operational amplifier A and the second operational amplifier A′ are preamplifiers, and are disposed in the seat ring 10 for collecting the ECG signals from the human body. The second stage operational amplifier A2 is configured to amplifying the differential signals transmitted by the first operational amplifier A and the second operational amplifier A′. The amplifier circuits of the two stages are separated and connected with the signal transmission lines, and the negative feedback resistor R₄ is replaced by two resistors R₄ and R₄′ that are then respectively provided in the signal acquisition units on the left and right portions of the seat ring 10.

Specifically, referring to FIG. 5, one signal feedback line is provided in each of the conductor through-holes on the left and right portions of the seat ring 10, and the negative feedback resistors R₄ and R₄′ are connected to each other by two shielded cable segments. That is, the negative feedback circuit consists of the negative feedback resistors R₄ and R₄′ and the two shielded cables. Referring to FIG. 6, in another embodiment, the negative feedback resistors R₄ and R₄′ are connected via a signal feedback line, that is, the negative feedback circuit consists of the negative feedback resistors R₄ and R₄′ and one shielded cable segment.

According to the aforesaid configuration, the negative feedback resistors R₄ and R₄′ are connected via one or two shielded cable segments, such that the inverting input terminals of the two operational amplifiers that are provided with active electrodes and physically separated by more than 10 cm are connected via the shielded cable, thereby reducing the interference of external electrical signals to the signal acquisition unit and improving accuracy of the system in acquiring ECG signals from the human body.

In this embodiment, the weak electrical signal acquisition circuit can be applied to any variety of toilet seats, and it can collect ECG signals from the human legs when people use the toilet, and can also effectively extract the ECG signals even in dry winter.

### EXAMPLE 3

As shown in FIG. 7, the present disclosure further discloses a toilet seat 100 having the weak electrical signal acquisition circuit. The toilet seat 100 includes an aforesaid seat ring 10 fitted with the weak electrical signal acquisition circuit, and a foot rest 30. The toilet seat further includes a seat ring adjustment mechanism.

The foot rest 30 includes a foot support plate 31 and a foldable connection mechanism 32. The foldable connection mechanism 32 includes two connection assemblies on each side of the foot support plate 31. One end of each connection assembly is connected to an outer side of the seat ring 10, and the other end of the connection assembly is connected to the foot support plate 31.

Furthermore, the seat ring adjustment mechanism is provided under the seat ring 10, and is configured to lift a front end of the seat ring 10 by a certain angle when the height of the foot support plate 31 is lifted.

Specifically, the toilet seat 100 according to this embodiment may include both an ordinary toilet seat 100 without heating and flushing functions and a smart toilet seat 100 with the heating and flushing functions.

Specifically, the connection assembly includes multiple sections of connection arms that are pivotably connected to each other, such that the connection arms are telescoped or folded to allow the foot rest 30 to be folded and received on the toilet bowl.

Specifically, the seat ring adjustment mechanism adopts a mechanical linkage mechanism to control the angle of the lifted seat ring 10 by the foot support plate 31.

In this embodiment, the two connection assemblies consist of multiple sections of connection arms, such that the foot rest 30 can be easily extended and received. In addition, the weight bearing leg 33 has an adjustable length, which allows to adjust the height of the foot rest 30 accordingly and change the height difference between the foot rest 30 and the seat ring 10, and thereby facilitates the use of the elderly and children.

It shall be noted that the above description is only the preferred embodiments of the present disclosure, and is not intended to limit the present disclosure. For those skilled in the art, various modifications and changes can be made to the present disclosure. Any modification, equivalent replacement, improvement, and the like as made according to the spirit and principle of the present disclosure shall be regarded as within the protection scope of the present disclosure.

1. A weak electrical signal acquisition circuit, comprising two signal acquisition units that comprise two signal receiving electrode sheets and two operational amplifiers, wherein the two operational amplifiers form a differential circuit, the differential circuit further comprising a first feedback resistor R₃, a second feedback resistor R₃′, and a negative feedback resistor R₄ provided between the first feedback resistor R₃and the second feedback resistor R₃′, wherein the negative feedback resistor R₄ serves as a negative feedback circuit for filtering differential mode signals received by the signal receiving electrode sheets.

2. The weak electrical signal acquisition circuit of claim 1, wherein the two signal receiving electrode sheets comprise a first signal receiving electrode sheet and a second signal receiving electrode sheet, the two operational amplifiers comprise a first operational amplifier A and a second operational amplifier A′, and the two signal acquisition units comprise a first signal acquisition unit and a second signal acquisition unit, wherein
the first signal receiving electrode sheet and the first operational amplifier A form the first signal acquisition unit, the first signal receiving electrode sheet being connected to a non-inverting input terminal of the first operational amplifier A, and the first feedback resistor R₃ being provided between an inverting input terminal and an output terminal of the first operational amplifier A; and
the second signal receiving electrode sheet and the second operational amplifier A′ form the second signal acquisition unit, the second signal receiving electrode sheet being connected to a non-inverting input terminal of the second operational amplifier A′, and the second feedback resistor R₃ ^{′} being provided between an inverting input terminal and an output terminal of the second operational amplifier A′.

3. The weak electrical signal acquisition circuit of claim 1, wherein the two signal receiving electrode sheets are active electrode sheets, and are configured to receive common mode signals and the differential mode signals.

4. The weak electrical signal acquisition circuit of claim 1, wherein the negative feedback circuit is formed by using a single resistor or a network assembly consisting of a plurality of electronic components and having a frequency characteristic;
or the negative feedback circuit comprises a network assembly having a frequency characteristic of full pass, low pass, high pass, band pass, or band resistance.

5. A seat ring having a weak electrical signal acquisition circuit, wherein the seat ring is fitted with the weak electrical signal acquisition circuit of claim 1.

6. The seat ring of claim 5, wherein the seat ring has two mounting holes respectively provided near tops of upper surfaces of left and right portions thereof, with the two signal acquisition units respectively provided in the two mounting holes, wherein the two mounting holes have openings respectively covered by the two signal receiving electrode sheets, with the first operational amplifier A and the second operational amplifier A′ respectively mounted in the two mounting holes.

7. The seat ring of claim 6, wherein the seat ring has two conductor through-holes respectively provided in the left and right portions, the two conductor through-holes having one ends provided respectively at bottoms of the two mounting holes and the other ends each provided at a connection portion between the seat ring and a seat lid, wherein
the two conductor through-holes have two signal transmission lines respectively provided therein, the two signal transmission lines having one ends respectively connected to output terminals of the first operational amplifier A and the second operational amplifier A′ and the other ends each connected to a secondary differential amplifier circuit.

8. The seat ring of claim 7, wherein each of the two signal transmission lines is in a form of a shielded cable.

9. A toilet seat having a weak electrical signal acquisition circuit, comprising a seat ring having a weak electrical signal acquisition circuit according to claim 5, wherein the seat ring toilet seat further comprises a seat ring adjustment mechanism that is provided under the seat ring and configured to lift a front end of the seat ring by a certain angle.
